(19) United States
(12) Patent Application Publication
(10) Pub. No.: US 2010/0016778 A1
(43) Pub. Date: Jan. 21, 2010

(54) APPARATUS FOR PURIFICATION OF BLOOD AND A PROCESS THEREOF

(76) Inventor: Budhaditya Chattopadhyay, Bangalore (IN)

Correspondence Address: FOLEY AND LARDNER LLP;SUITE 500, 3000 K STREET NW, WASHINGTON, DC, 20007, US

(21) Appl. No.: 12/310,317

(22) PCT Filed: Aug. 16, 2007
(86) PCT No.: PCT/IN2007/000347

§ 371 Date: Sep. 25, 2009

(30) Foreign Application Priority Data
Aug. 23, 2006 (IN) 01502/CHE/2006

(51) Int. Cl. A61M 1/16; B01D 65/02; B01D 61/28; B01D 61/54; B23P 11/00

(52) U.S. Cl. 604/6.09; 210/636; 210/188; 210/137; 210/109; 29/428

(57) ABSTRACT

The present invention is in relation to an apparatus for purification of blood and a method for assembling such apparatus. Also, the invention provides solution to the problem of kidney damage, wherein the damaged kidney can be replaced with the apparatus of instant invention to help in the purification of blood and thus helps the patients at large having kidney related disorders.

### FIELD OF THE INVENTION

The present invention is in relation to the field of purification of blood. More particularly, the present invention provides an apparatus for purification of blood and a method of assembling such apparatus. In addition, the invention also provides a method of placing an apparatus inside the body or a portable device which would be externally attached with the subject.

### BACKGROUND OF THE INVENTION

Kidney, whose function is the elaboration and excretion of urine, consists of approximately one million nephrons compose each bean-shaped kidney. The filtration unit of the nephron, called the glomerulus, regulates the concentration within the body of important substances such as potassium, calcium, and hydrogen, and removes substances not produced by the body such as drugs and food additives. The filtrate, urine, leaves the nephron through a long tubule and collecting duct. Chemical signals triggered by the body's need for water and salt cause the walls of the tubule to become more or less permeable to these substances, which are reabsorbed accordingly from the urine.

We are all aware that kidney is a pair of bean shaped vital organs of human body which excrete metabolic waste products in the form of urine and hence balances the body chemistry by purifying blood. The kidneys lie on the posterior abdominal wall, one on each side of the vertebral column, behind the peritoneum and bellow the diaphragm, extended from 12^{th} thoracic vertebra to 3^{rd} lumber vertebra. Each kidney consists of one million nephrons which are called as the unit of kidney. Kidney regulates the concentration of important substances such as potassium, sodium, etc and removes substances which are not produced by the body such as drugs, food additives, along with metabolic waste materials of the body like urea, uric acid, cratinine, phosphate etc. Apart from purification of blood, ERYTHROPOIETIN is released by specialized cells found in the kidney, in response of hypertoxia. Erythropoietin is a major stimulus for the production of Red Blood Corpuscle (RBC) in bone marrow. It also regulates water and electrolyte balances, body fluid as molality and electrolyte concentrations. Arterial pressure and acid-base balance are also regulated by kidneys. Kidney failure means degradation in ideal operational efficiency than that in normal ideal condition. Often, in some cases, kidney does not function properly; where we need to go for dialysis (haemodialysis, peritoneal dialysis) or kidney transplantations. There are several reasons and diseases leading to kidney malfunctioning or kidney failure, with salient symptoms according to diseases.

In humans, kidneys are situated one on each side of the spine, and are embedded in fatty tissue. They are bean-shaped, possessing a convex outer border and a concave inner border. The inner border presents an indentation, the hilum, at which the blood vessels enter and leave. In front is the renal vein carrying blood from the kidney; behind it lies the renal artery carrying blood to the kidney. Most posterior is the ureter, a tube that conveys urine to the bladder. The hilum arises from a deeper indentation, the sinus of the kidney, in which the ureter dilates to form a small sac, the renal pelvis. The kidney also embodies glomeruli, aggregations or loops of capillaries enclosed within thin envelopes of endothelial lining called Bowman's capsules, located at the blind ends of the renal tubules

Urine is produced in the glomeruli and renal tubules and carried to the renal pelvis by collecting tubules. The glomeruli act as simple filters, through which water, salts, and waste products from the blood pass into the spaces of Bowman's capsules and from there down into the renal tubules. Most of the water and salt is reabsorbed from these tubules; the remainder is excreted as urine. The renal tubules also secrete other salts and waste products from the blood into the urine. The average amount of urine excreted in 24 hours is about 1.4 liters (2.4 pt), but the quantity varies considerably, depending on intake of fluid and loss from such sources as the skin in perspiration, or from vomiting.

The kidneys are also important in maintaining a balance of fluid and salt and a normal degree of acidity. When disorders upset these delicate balances, the kidneys act to restore them by excreting more or less water, salt, and hydrogen ions. The kidneys help maintain normal blood pressure by secreting the hormone renin and elaborate a hormone that stimulates the production of red blood cells.

Human Kidney for Transplant A surgeon removes a donated kidney from its shipping container, where it is maintained in saline solution and packed in ice. A single kidney is sufficient to keep its recipient healthy because it will enlarge to function for the whole body. Kidney transplants are more straightforward than heart, liver, or lung transplants and 80 to 90 per cent are successful. If the kidney is rejected, the patient can return to dialysis and, if otherwise healthy, undergo a second transplant operation.

It is well known that kidney functions on several processes of physical chemistry like dialysis, diffusion, filtration and ultra purification of blood, etc.

Nephritis, or inflammation of the kidney, is one of the commonest kidney diseases. Its chief characteristics are the appearance in the urine of such elements as albumin, a condition known as albuminuria; red and white blood cells; and hyaline or granular casts, all revealed by microscopic examination of the urine. It is much more common in childhood and adolescence than in middle age.

The commonest form of nephritis is glomerulonephritis; it often occurs within three to six weeks following a streptococcal infection. The patient complains of chills; fever; headache; backache; puffiness, or oedema, of the face, especially around the eyes; nausea; and vomiting. Urine may become scanty and smoky in appearance. Prognosis is generally good, and most patients recover completely. A few people, however, develop chronic nephritis. In this form of nephritis, kidney damage progresses over many years, during which patients are symptom free. Eventually, however, they may develop uremia (urine in the blood) and kidney failure.

The nephrosis includes a variety of types of nephritis marked by degenerative changes in the tubules of the kidney. Pure nephrosis is rare; more common are those types associated with glomerulonephritis or other diseases affecting the kidney. Nevertheless, the term nephrosis is still employed for a syndrome characterized by the presence of generalized oedema, by large amounts of albumin in the urine, by excessive cholesterol in the blood, and by relatively normal urinary output.

Nephrosclerosis, or hardening of the small arteries supplying the kidney, is a disorder characterized by the presence of albumin, casts, and occasionally white or red blood cells in the urine (haematuria); it usually accompanies hypertensive vascular disease. Its fundamental lesion is a sclerosis of the small arteries of the kidney, with secondary atrophy of the glomeruli and pathological changes in the interstitial tissue.

Renal calculi, or kidney stones, may form in the kidney or renal pelvis from crystals deposited from the urine. They are composed mostly of calcium oxalate. Infection or obstruction may play a part in their formation. Sometimes they occur when the level of blood calcium is abnormally high, as may be the case when the parathyroid glands overproduce urine. Occasionally, stones may develop when the blood level of uric acid is too high (i.e. Gout), usually from over consumption of meat. Excessive dietary intake of calcium and oxalate and low fluid intake have also been associated with formation of stones. In most cases, however, the cause is not known. Stones may cause bleeding, secondary infection, or obstruction. Small kidney stones tend to travel down the ureter towards the bladder; their movement is usually accompanied by severe pain. Colic caused by stones usually requires one or more injections of painkilling drugs for relief. The pain may develop suddenly after muscular exercise. Once a stone drops into the bladder, it may be passed with the urine unnoticed, and the pain ceases. If the stone is too large to pass, treatment is necessary, either with surgery or with lithotripsy, a procedure that uses shock waves generated outside the body to disintegrate the stones.

Uremia is poisoning caused by accumulation in the blood of waste products normally excreted by the kidney. It occurs most often as the end stage of chronic kidney disease and is characterized by drowsiness, headache, nausea, inability to sleep, spasms, seizures, and coma. Prognosis is poor. By the 1980s, however, such techniques as repeated periodic dialysis to clear the blood of accumulated waste products and toxins, and kidney-transplant operations, offered new hope to patients. Kidney diseases are mainly classified in two categories: Treatable (medication/surgery of subject) and Non-treatable (renal transplantation using donor's kidney). Though now a days, renal transplantation is a common treatment/surgery, but the subject cant sustain for a long time with the transplanted foreign kidney of the donor's. Such transplantation requires blood group, tissue matching and other biomedical (chemical) checkup of both the donor as well as acceptor, if every parameters are matched properly then only we can go for a transplantation. But here also our body rejects the new kidney. Initially doctors prescribes some medicines for ease in acceptability of donor's kidney, but such medicines can't be continued in long run (maximum 5-8 years properly), because of its excessive side effects. Again, the subject needs to wait for a donor.

The present scenario suggests that the existing dialysis processes are not able to overcome all the defective parameters of blood as well as body because of kidney failure as defined.

Ultimately in case of renal failure, subject needs to go for kidney transplantation with a donor's kidney. However, for this purpose, there are several parameters of body chemistry to be matched with donor's functional kidney. Here we are aware that the success rate of kidney transplantation is not very encouraging. Sometimes in this case body treats the transplanted kidney as foreign material and tries to reject it. It is clear that for the proper functioning of the natural kidney, the complete pair is in functional state. However it also works in ½ active redundancies i.e.; the donor after donating one-out-of-two kidneys to the acceptor can also retain good health. Now it is over stressed kidney system for both donor and acceptor. This leads to increased failure rate and high risks to working kidney in future. In other words, donor's life is now at great risk for mere sympathy. Besides this, any minor clinical procedure for single kidney system becomes threat to life and equally difficult in the procurement of donor's natural kidney for transplantation from open world market.

Pyelonephritis is an infection of the kidney with bacteria. Acute pyelonephritis is often accompanied by fever, chills, pain on the affected side, frequent passing of urine and burning on urination. Chronic pyelonephritis is a progressive, usually symptom-free disease that may eventually lead to destruction of the kidney and to uremia. Pyelonephritis is more common in women than men, and more usual in diabetics.

Kidney diseases are also classified as End Stage Renal Disease and Acute Stage Renal Disease. In this disease conditions, the workability of the kidney is lost. Henceforth, there is a need to take care of this disease condition. Thus, the solution for all this kidney related problems can be provided using the application of instant invention.

Wilms's tumour, a highly malignant form of kidney tumour, is most frequent in young children. Recently devised treatment has brought about a cure in many children with this disease. In systemic lupus erythematosus, which tends to strike women in their thirties more than other groups, the body makes antibodies that damage the kidney.

Now it is really good news for the entire humanities where the inventor of this artificial kidney called Transplantable Artificial Kidney (TAK) has successfully realized the device to overcome all those problems associated with the treatment of malfunctioning of kidney either through dialysis or by transplantation with donor's natural kidney. That is also with great ease and at no cost compared to the risk and high cost of availability of stressed kidney transplantation adopted for remedial measures from kidney related diseases.

The most encouraging part of this wonder kit lies in the fact that it is a universal kit, applicable; irrespective of blood group, tissue matching and any other investigations related to kidney transplantation. It gives satisfactory results beyond expectations. Lastly the greatest pleasure is derived from the fact that it gets rid of sacrificing donors. Hence it's a requirement of us to have an alternative like artificial kidney, what will be able efficiently to meet the job of the kidney.

### PRIOR ART FOR THE PRESENT INVENTION

Now a days the dialysis membrane available is use and throw type, means once/twice after its application it won't be reusable. The normal dialysis system is totally external system, which can't be placed in our body. For the dialysis of patient the doctor, nurse hospital is required, here for the first time to set it into the body the doctors or the hospital is required.

Normal dialysis system always has some risk of air-bubble entering into blood which can take the life of the patient within no time. But in artificial kidney, there is no chance to enter the air bubble in the blood.

The normal dialysis system purifies the blood after some period, not in continuous basis, so in other way it harms the cells of our body by existence of the metabolic materials obtained after metabolism in cells.

But using the artificial kidney we can able to make the purification of blood an a continuous process and hence it wont affect our health and the physical system will be normal only. The normal dialysis is painful and even can't be done properly; but once artificial kidney will be fitted in the body of the patient he will be all right. We can replace the kidney with the artificial kidney system.

The related art of interest describes various methods for purification of blood, but none discloses the present invention. The related art will be discussed in the order of perceived relevance to the present invention.

U.S. Pat. No. 3,864,259

In this document efforts have been made to help patients suffering from kidney malfunctioning and calling for dialysis by eliminating the immobility of the patients and making the dialysers portable & attachable to the patient's body under the clothing as described in this patent. However the method is not at all able to provide proper/sufficient clearance of metabolic wastes from blood. Further more excess water in blood causes EDEMA for subject. Thus, the Applicant is able to overcome the disadvantages associated with the aforementioned citation.

U.S. Pat. No. 4,354,933

The physical-chemical feasibility of this invention raises serious doubts. The composition of the described dialyzing fluid has an oncotic pressure which enables the dialyzing fluid to extract water and other dissolved materials from blood but does not enable it to enriching the same. Hence it's doubtful what the venous blood could receive from the dialyzing bath. However, this patent is no where in relation to the application of instant invention and the instant invention helps in overcoming the disadvantages of the patent.

U.S. Pat. No. 5,092,886

The aforementioned US patent makes use of ultra-filtration only and there is no dialysis occurring. In addition, it is evident from the document that the diffusion rate is very slow. In conclusion, this document uses a system which is only able to do ultra-filtration leading to excessive water loss from blood, and comparatively increase of the metabolic waste products, ultimately it results in misbalance in body chemistry of the subject which would surely be observed after the usage of apparatus. However, in the present invention the Applicant provides solution to all the aforementioned problems of the aforementioned citation.

Till date there is no polymeric dialysis membrane sandwiched in Nano-carbon net. Polymeric membrane reinforced nano-carbon/other nano metallic (treated for bio-compatibility) net was also never used in medical dialysis system. The “V” shaped polymeric membrane (with or without nano-carbon/nano-metallic or bio-compatible nylon net or mesh) helps in increasing blood pressure as the blood moves through the membrane path as described in the detailed description of the specification.

The present invention provides in the apparatus a provision for Refreshment/dialysis cycle for the membrane and hence increasing the life time of them. In addition, it makes use of L1 and L2 solution to attain its optimum efficiency at the same time balancing body chemistry. Also, L1 & L2 compositions are also variable according the condition of subject. Valves help in controlling the dialysis and refreshment cycle. Control system helps in circulator for super clearance of wastes. “S” or “V” or any other shaped pumps/circulators can be provided to make the system more efficient in means of increased clearance rate of metabolic wastes as well as blood pressure control. Also, there is portability if attached externally in some subject. Further there is a provision for the variable clearance of the metabolic wastes as well as water according the condition of the subject.

None of the above citations, taken either singly or in combination, is seen to describe the instant invention as claimed. Thus, obtaining apparatus of instant invention using the method of instant invention will therefore helps in addressing the problems associated with the prior art. The novelty resides not only in the apparatus but also the process of purifying the blood using instant invention. It is the sequence of steps involved which are unique and which has resulted in arriving at, pure blood in case of kidney damage.

### OBJECTS OF THE PRESENT INVENTION

The principal object of the present invention is to develop a medical apparatus. Another object of the present invention is to develop an artificial kidney or apparatus for purification of impure blood. Yet another object of the present invention is to develop a method for assembling of artificial kidney. Still another object of the present invention is to provide a method for purification of blood using the apparatus of instant invention. Still another object of the present invention is to develop a method for positioning the apparatus in a subject. Still another object of the present invention is to bring about purification of blood. Still another object of the present invention is to send the urine to the bladder.

### STATEMENT OF THE INVENTION

Accordingly, the present invention provides an apparatus for purification of blood, wherein said apparatus comprising membranes (16, 17) placed inside the chambers (15, 18) respectively, wherein chambers (15, 18) are located adjacently; an impermeable jacket (9) having porous permeable membrane (9A) is connected to membranes (16, 17) through channel (11); unidirectional valve (8) is connected to the porous permeable membrane (9A) to allow unpurified blood into the membranes (16, 17) using the channel (11); a chamber (26) is connected to membranes (16, 17) through channel (13) to store L1 solution provided with bubble trapper valve (25) to prevent entry of air bubbles into the chamber (26); outlets of chambers (18, 15) are connected to waste outlet (19) through channel (19A, 19B) respectively to carry impure L2 solution; and outlets of membranes (17,16) are connected to purified blood outlet (20) through channels (20A, 20B) respectively to circulate purified blood to veins; a method of assembling an apparatus for purification of blood, wherein said method comprising steps of: placing membranes (16, 17) in chambers (15, 18) respectively; connecting impermeable jacket (9) having porous permeable membrane (9A) to the membranes (16, 17) through channel (11); connecting chamber (26) to the membranes (16, 17) through channel (13); connecting outlets of the chambers (18, 15) to waste outlet (19) through channel (19A, 19B) respectively and connecting outlets of the membranes (17,16) to purified blood outlet (20) through channels (20A, 20B) respectively; and mounting valves (4, 5) on to said channel (19A, 19B) respectively to control flow of waste and fixing knobs (1, 1A) to control flow of unpurified blood through channel (11) and purified blood through channels (20A,20B); and a method for purification of blood, wherein said method comprising steps of: allowing impure blood to undergo coarse filtration/purification/dialysis through porous permeable membrane (9A) to remove the waste through outlet (19); directing coarse filtered impure blood using knob (1) into one of the membranes (16 or 17) to undergo filtration while refreshing the other membrane (16, or 17) using L1 and L2 solution at a given time; and collecting waste material into L2 solution after filtration and thereby removing the waste through the outlet (19) to obtain purified blood in outlet (20).

### BRIEF DESCRIPTION OF THE ACCOMPANYING DRAWINGS

FIG. 1 Diagram of artificial kidney showing the complete apparatus of the instant invention

FIG. 2 Shapes of the membranes

FIG. 3a Shape of the system from front view

FIG. 3b Shape of the system from side view

FIG. 4 Catheter for connecting blood vessels to the apparatus

FIGS. 5A, B, C, D, and E Discloses the control system for various valves

FIG. 6: Cellulose acetate membrane sandwiched between the nano-carbon nets

### DETAILED DESCRIPTION OF THE INVENTION

The present invention is in relation to an apparatus for purification of blood, wherein said apparatus comprising membranes (16, 17) placed inside the chambers (15, 18) respectively, wherein chambers (15, 18) are located adjacently; an impermeable jacket (9) having porous permeable membrane (9A) is connected to membranes (16, 17) through channel (11); unidirectional valve (8) is connected to the porous permeable membrane (9A) to allow unpurified blood into the membranes (16, 17) using the channel (11); a chamber (26) is connected to membranes (16, 17) through channel (13) to store L1 solution provided with bubble trapper valve (25) to prevent entry of air bubbles into the chamber (26); outlets of chambers (18, 15) are connected to waste outlet (19) through channel (19A, 19B) respectively to carry impure L2 solution; and outlets of membranes (17,16) are connected to purified blood outlet (20) through channels (20A, 20B) respectively to circulate purified blood to veins.

In another embodiment of the present invention wherein the flow of L2 solution into the chambers (15,18) is controlled using knobs (2, 3) respectively.

In yet another embodiment of the present invention wherein knob (1) is used to control the flow of impure blood and L1 solution into the membranes (17, 16).

In still another embodiment of the present invention, wherein said knob (1) allows either the impure blood or the L1 solution into a membrane (17, 16) at any given time.

In still another embodiment of the present invention, wherein said knob (1) maintains impure blood in any one of the membranes (17, 16) and L1 solution in the other membrane (17, 16).

In still another embodiment of the present invention, wherein knob (1A) is used to control the flow of pure blood from the membranes (17, 16) through the channels (20A, 20B) to the outlet (20).

In still another embodiment of the present invention, wherein said knob (1A) opens one of the channels (20A, 20B) at any given time.

In still another embodiment of the present invention, wherein valves (4, 5) are used to control the flow of L2 solution from the chambers (15, 18) into the outlet (19) through channels (19A, 19B).

In still another embodiment of the present invention, wherein valve (6) controls the removal of waste collected in impermeable jacket (9) through the outlet (19).

In still another embodiment of the present invention, wherein said membranes (16, 17) are biocompatible and are made up of polymers selected from a group comprising polyvinyl halides, polyurethanes, polystyrene derivatives, polyolefins, polyester series condensates, cellulose series high polymers and combinations thereof.

In still another embodiment of the present invention, wherein said membranes (16, 17) are preferably made up of polyurethanes selected from a group comprising segmential polyurethanes and polyurethane urea.

In still another embodiment of the present invention, wherein said polymer membranes (16, 17) are sandwiched between single layered/multi layered nano-carbon/any other bio-compatible nets.

In still another embodiment of the present invention, wherein said polymer membranes (16, 17) are placed in “V” shape.

In still another embodiment of the present invention, wherein said channel (11) is divided into two sub channels (11A, 11B) to connect membranes (17, 16) respectively. The present invention is in relation to a method of assembling an apparatus for purification of blood, wherein said method comprising steps of: placing membranes (16, 17) in chambers (15, 18) respectively; connecting impermeable jacket (9) having porous permeable membrane (9A) to the membranes (16, 17) through channel (11); connecting chamber (26) to the membranes (16, 17) through channel (13); connecting outlets of the chambers (18, 15) to waste outlet (19) through channel (19A, 19B) respectively and connecting outlets of the membranes (17,16) to purified blood outlet (20) through channels (20A, 20B) respectively; and mounting valves (4, 5) on to said channel (19A, 19B) respectively to control flow of waste and fixing knobs (1, 1A) to control flow of unpurified blood through channel (11) and purified blood through channels (20A,20B).

In another embodiment of the present invention, wherein knob (2, 3) are placed to control flow of L2 solution through channel (12) which is connected to chambers (15, 18).

In yet another embodiment of the present invention, wherein valves (27, 28) are placed to control flow of L1 solution into membranes (17,16) through sub channels (11A, 11B).

In still another embodiment of the present invention, wherein said polymer membranes (16, 17) are placed in “V” shape.

In still another embodiment of the present invention, wherein said polymer membranes (16, 17) are sandwiched between single layered nano-carbon nets.

The present invention is in relation to a method for purification of blood, wherein said method comprising steps of: allowing impure blood to undergo coarse filtration through porous permeable membrane (9A) to remove the waste through outlet (19); directing coarse filtered impure blood using knob (1) into one of the membranes (16 or 17) to undergo filtration while refreshing the other membrane (16, or 17) using L1 and L2 solution at a given time; and collecting waste material into L2 solution after filtration and thereby removing the waste through the outlet (19) to obtain purified blood in outlet (20).

In another embodiment of the present invention, wherein the filtration of blood involves removal of waste such as urea, uric acid, creatinine and other metabolites.

In yet another embodiment of the present invention, wherein the refreshment of membranes (16 and 17) involves removal of impurities which are blocked inside the membranes (16 or 17).

In still another embodiment of the present invention, wherein the refreshment of membrane (16 and 17) involves maintaining L1 solution inside the membrane (16 and 17). and L2 solution outside the membrane (16 and 17) but inside the chamber (15, 18). In still another embodiment of the present invention, wherein said L1 solution is purified water with glucose and L2 solution is a mixture of sodium, potassium, chloride, calcium, magnesium, acetate/citrate, bicarbonate, glucose along with drugs belonging to the class of antiplatelets, anticoagulants, antifibrins, antithrombins, antiproliferatives, antiplatelets, anticouagulants, antifibrins, antithrombins and combinations thereof.

In still another embodiment of the present invention, wherein said L2 solution has density always maintained higher than that of L1 solution to create back pressure on the membrane (16 or 17) so as to remove the blocked impurities.

In still another embodiment of the present invention, wherein the membrane (16, 17) are sandwiched between single layered nano-carbon nets.

In still another embodiment of the present invention, wherein the membrane are placed in “V” shape to create blood pressure and to achieve rapid filtration of blood.

The technology of the instant Application is further elaborated with the help of following examples. However, the examples should not be construed to limit the scope of the invention.

### Example: 1

Membrane 1

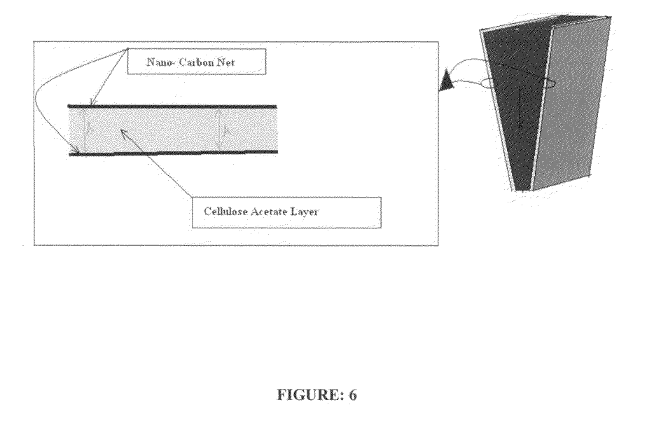

The membrane is developed by sandwiching two very thin smooth fiber membrane which is made up of carbon based nylon string or such bio-compatible materials which wont react with blood and wont harm our health also, and each fiber should be of 2 micron diameter preferably (or lesser/higher than the above mentioned diameter); with the inner layer as cellulose acetate or a cellulose acetate derivatives (or any other bio-compatible polymers/mixture of them); and this middle layer is very thick compare to the two outer layers which is as shown in FIG. 6.

Here we prefer to make the outer fiber layer to be very thin so that the bloods can come to the contact of the membrane. When we shall make a single layer densed-compressed net with such fiber, the small gaps in the net should be lesser than 2 micron (or greater than that). The fiber should be smooth enough so that it won't make any kind of resistance in the flow of blood and also not damage the living blood cells. As the blood cells like WBC, RBC, etc having the diameter greater than 2 micron, at any cost no blood cells will be able to cross the membrane. Hence it will become a protection to our blood circulation system, with the guarantee that not a single living blood cell is going to loose from our body.

The two outer lairs of membrane can be formed by making the wholes of 2 micron in some continuous non-fibric/fibric very thin {i.e 2 micron} sheet made of the materials which wont react with blood and wont be hazardous in long run.). The urea, uric acid, creatinine and some other unwanted materials will be only able to cross the semi-permeable cellulose acetate inner membrane. The crossing of urea and other metabolic elements through the cellulose membrane takes place by dialysis process.

### Example: 2

Membrane 2

Else we can make a membrane by taking two thick membrane sheets made of cellulose acetate or cellulose acetate derivatives, and provide a very small gap between these two membranes. The gap provided will be the path to flow the blood inside the bilayer membrane made of cellulose acetate or cellulose acetate derivatives or other bio-compatible polymer or their mixture, or any other bio-compatible materials, and the gap between the two bilayer membranes should be descending in order.

Using the membrane (1 or 2) we first made a sheet of membrane. Then we take such two sheets of membrane and prepare a new model of it by making a bilayer membrane providing some gap/channel between such two layers so that blood can circulate through this path. Here we should be careful so that the channel inside of the bilateral membrane should be in “v” shape, means the gap between two layers of membrane should be in decrement in order. It will bring more and more polluted bloods in contact with the membrane, so that dialysis can takes place in a very fast phase by increasing the contact surface of the polluted blood and the other solution “L2” placed outside the membrane through dialysis, via semi permeable membrane, provided to the system.

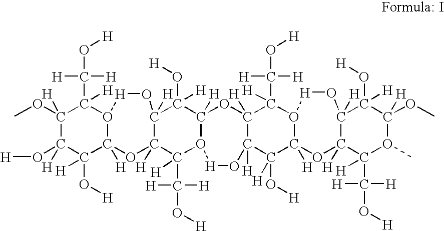

Formula: I represent the structure of the Cellulose Acetate (membrane) chain, it's the inner layer for TYPE 1 membrane; and for type 2 membrane it can be used directly.

The various materials used for making membrane are as follows:
Cellulose acetate, cellulose diacetate, cellulose tri-acetate, (regenerated cellulose, cuprammonium cellulose, cuprammonium rayon, saponified cellulose ester,) etc.
Polyacrylonitrile(PAN), polysulfone, polycarbonate, polyamide, polymethyl-methacrylate (PMMA), polyurethane, etc.
Conventional coated membrane with anti-oxidant substances such as Vitamin E, anticoagulant or any other drugs coated membrane, etc.
Polyethersulfone, Polyvinylpyrrolidone polyamide, polytetrafluroethylene, silicones, fluroethylpolypropylene, polypropylflurinated amines, (other fluorinated polymers), membranes with anti coagulant and other agents, etc.
Materials/membranes can also be used with very high efficiency as stated in US patent “US 2004/0200991 A1; U.S. Pat. No. 4,957,508; U.S. Pat. No. 5,749,880, etc.” other conventional membranes or the bio-compatible membranes which may be developed in future.

Preparation of the Membrane:

Initially, the liquid polymeric solution is poured in mould according the desired shape of membrane, then the using appropriate dies, we get the desired shape of membrane. Now, the obtained membrane is sandwiched in the nano-carbon nets. Here the nano-carbon net has higher aperture size than the size of the pours of the polymeric membrane.

In this step we heat the total (complete) compound to a higher temperature till the polymer becomes sticky/semi liquid condition, for very short time. Now the total assembly is cooled. This is the first desired membrane. The other wall of the membrane can be prepared accordingly; the only condition is care should be taken while preparing mould. Like in case of preparing semi spherical membrane, the inner membrane might have the radius of 2 cm (greater/lesser than that) (with the specified wall thickness), so its outer membrane must have higher radius than that of the inner one.

There are several conventional and advanced processes (even many new process might come in coming future to prepare desired membrane) to prepare reinforced polymer membrane. Like in case of medicated blood stain/blood vessels a metallic mesh/net/other structure are coated with polymers. There the aperture/gap in metallic structure is very low. But while preparing the membrane for the present invention, we make the nano-carbon/nano-metallic net with much larger aperture size so that the metabolic wastes can cross through the polymeric membrane pores, and the net inside the polymeric membrane is able to provide higher strength as well as able provide higher life time. Now this reinforced polymeric membrane can be used as a unit membrane or can be sandwiched between two nano-carbon net. The polymeric membrane can also be used directly. The shape of membranes is parallel plate membrane, can also be used as “V” shaped membrane or any other shape (like hollow fiber membrane with conical shaped hollow in it/general conventional hollow fiber membranes; coil type membranes, etc.). It can be achieved by proper moulding & dieing techniques, as well as adhesion, annealing technology. No bio-hazardous/bio-incompatible materials should be used for preparing membranes, if used care should be taken to remove them properly before application. Here preferably we use SINGLE LAYER nano-carbon/nano-metallic net rather than double layer nano-carbon/nano-metallic membrane.

### Example: 3

Shape of the Membrane

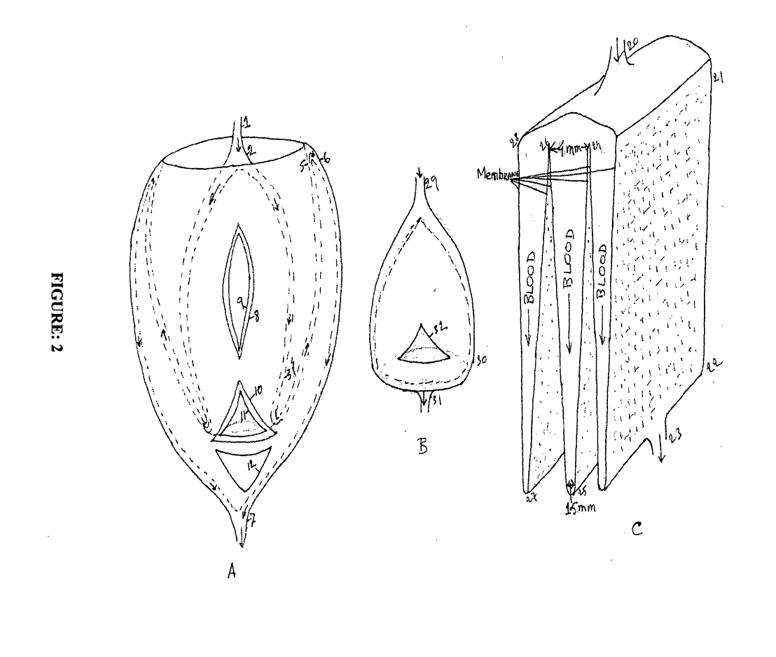

The semi spherical (Bowman's capsule shaped FIG. 2A, B) membrane have the highest efficiency because of its sudden change in pressure, higher surface area, higher contact surface/unit volume, etc. Here it can be observed from this figure, some amount of blood is entering to the membrane (FIG. 2, A, B, C) at the points 1, 29, 20 respectively (suppose for understanding; the volume is 10 ml), then it follows the path of the membranes accordingly as shown in figures. It is very clear that the surface area of the membrane is increasing continuously though the volume of blood is constant. So it's enhancing the contact surface of blood. Again, the pressure of blood keeps on changing because of the “V” shaped/walled membrane. It is observed that the error materials like urea, creatinine, uric acid, phosphates, etc are becoming suspended particle. So it is quite easier to get them dialyzed, and bring them out from blood flow. Here the pressure is obtained because of blood flow and shape of the membrane Apart from the FIG. 2; A, B, C the membrane can be of any other shape too.

The membrane is preferred to be in “V” shaped or Parallel plate shape. Here the V shaped membrane has maximum efficiency level because of its increased pressure parameters. The blood flow through the membrane is “V” shaped, so the pressure is always increasing/altering.

Pore Size:

Radius: approximately 0.02-2 μm (preferably bellow 1.2 μm); the pore size may be larger/smaller than that of the specified.

Porosity: 50-85% (lesser/greater than that of the specified)

Wall Thickness: Approximately 5-60 μm, and can be much or less thick/thin than the mentioned specification.

### Example: 4

Actual Surface area of membrane: 0.03-1 mm² (it might be larger/smaller than this)

For testing membrane efficiency compared to conventional membrane, a symmetrical large membrane (surface area=1.6 m²) was prepared:

Sterilization: Electron Beam/steam/gamma irradiation, etc.

Test: 1

Performance: With Carbon Net/(Bio-Compatible/Inert) Metallic Net:

| | |
|---|---|
| K_{Uf} mL/hour/mm Hg: | 135 |
| Urea Clearance (Q_{B} = 200 ml/minute): | 321 |
| K₀A mL/minute: | 2210 |
| Priming Volume mL: | 165 |

Test: 2

Performance: Without Carbon Net (Bio-Compatible/Inert) Metallic Net:

| | |
|---|---|
| K_{Uf} mL/hour/mm Hg: | 110 |
| Urea Clearance (Q_{B} = 200 ml/minute): | 305 |
| K₀A mL/minute: | 2010 |
| Priming Volume mL: | 156 |

Here, Kuf=Ultra filtration coefficient, KOA=mass transfer area coefficient for urea QB=Blood flow rate.

### Example: 5

Preparation of L2 and L1 Solutions

The L2 Solution Can Be Prepared By Using the Following Materials as Disclosed in Table 1

TABLE 1
List of ingredients and their concentrations
for preparation of L2 solution

| | | Concentration |
|---|---|---|
| Sl. No. | Component | (mM) |
| 1 | Sodium | 135-145 |
| 2 | Potassium | 0-4 |
| 3 | Chloride | 90-130 |
| 4 | Calcium | 1-1.2 |
| 5 | Magnesium | 0.2-0.3 |
| 6 | Acetate/citrate | 1-5 |
| 7 | Bicarbonate | 25-40 |
| 8 | Glucose | 0-15 |
| 9 | PCO₂ | 40-90 |
| 10 | pH | 7.1-7.3(units) |

Here we can also use antiplatelets, anticoagulants, antifibrins, antithrombins and other therapeutic drugs but the dosage is less than that in L1 solution. In case of the direct removal of the waste products after dialysis from the body, gel type alumina (containing 28% by weight of silica) (0.2 gm); activated carbon (0.5-8 gm) can also be applied in acute renal failure, where the metabolic waste clearance is required in a faster manner.

L1 Solution Consists of the Following Materials:

Distilled highly filtered and purified water with glucose, without any air bubbles or any other granular particles of any other molecules (as mentioned in table of Example:5; or a prescribed by doctors) is applied as L1 solution. Here we can use antiplatelets, anticoagulants, antifibrins, antithrombins and other therapeutic drugs as prescribed by doctors. Here we should remember the concentration of L2>>L1 so that proper back pressure can be created in it. This backpressure on the membrane refreshes the membrane uniquely.

Here the therapeutic drugs which gets stored in refreshment cycle leads an useful role in dialysis cycle. Though the membranes are made of biocompatible materials and embedded therapeutic drugs are also coated on the surface, but in some cases this coating layer might get removed. In that instant, the newly used/inserted therapeutic drugs would be stored in those vacant spaces; and hence the membrane would be able to use for long time. The concentration of these drugs should be determined by doctors, according the pathological reports. Dosage: Preferably as less as possible. These drugs may also be applied to L2 solution to increase its density.

### Example: 6

Construction and Working of Blood Purification Apparatus or Artificial Kidney

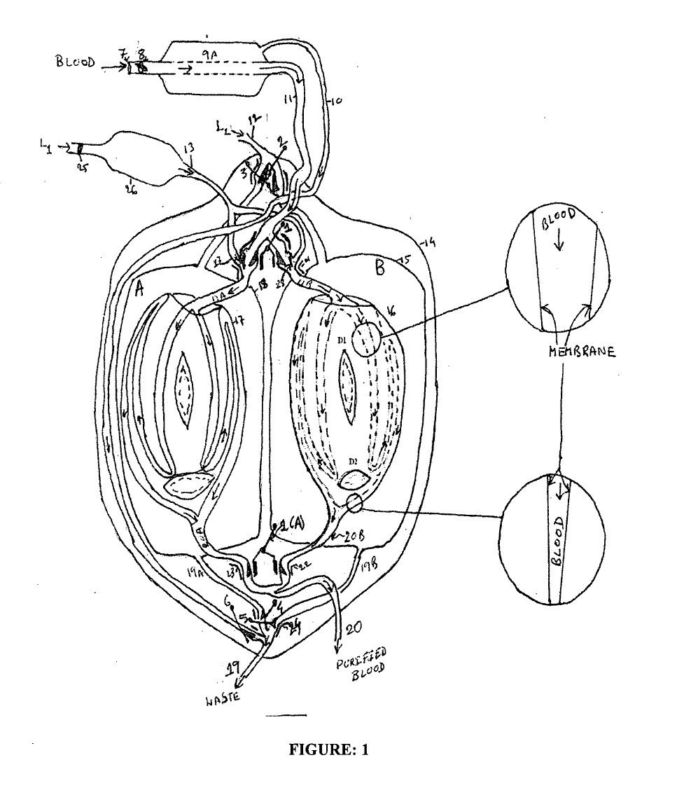

In FIG. 1, 7 provide the connection of the system to renal artery through which unpurified blood is entering the artificial system. 8 is an unidirectional valve (already existing in market, like heart valve; but smaller than that in size and shape) which allows unpurified blood to enter in the artificial system but cant go back through that. 8 is connected to a porous permeable bio-compatible membrane (9A) (vessel/channel) which is placed in a impermeable jacket (9), locked from both sides having a smaller path 10, through which ultra-filtrated water can comes out of 9. Here we should remember that 9 & 9A are not in touch with each other.

This total system can also be placed in the outer cover 14 (FIG. 1).9A allows Ultra-filtration of blood, which is again controlled by knob No: 6 of the same diagram. This knob allows the ultra-filtrated water to send out with other metabolic wastes. Again if required, it can be blocked by altering the position of knob 6, and hence excess water loss from blood can stopped immediately. After ultra filtration, blood goes through channel number 11, which is divided in two parts (11A, 11B) respectively. These two paths are again controlled by knob 1. Now the blood enters the membrane 16 & 17 in the respective chambers A & B. At a time unpurified blood either can go through 11A or 11B. Passing through the respective membranes of their corresponding path, blood comes out through the point 20. The dialysate solution L2 enters the chambers A & B respectively, which is controlled by knob 2 & 3. Knob 2 & 3 can also be replaced by some unidirectional valves, but in that case 2 entering paths for L2 solution is required. After dialysis, the metabolic wastes come out from the unpurified blood, and sent out from the system by the path 19A & B respectively. 19A is controlled by the knob 4; 19B is controlled by the knob 5. It can be observed that the channels 19A, 19B and 10 are meeting at a common junction point and after wards (metabolic wastes/urine) coming out from the artificial system through the outlet 19, which can either be connected to urinary bladder/the natural path leading to bladder (even in some cases in can be directly sent out externally from the system).

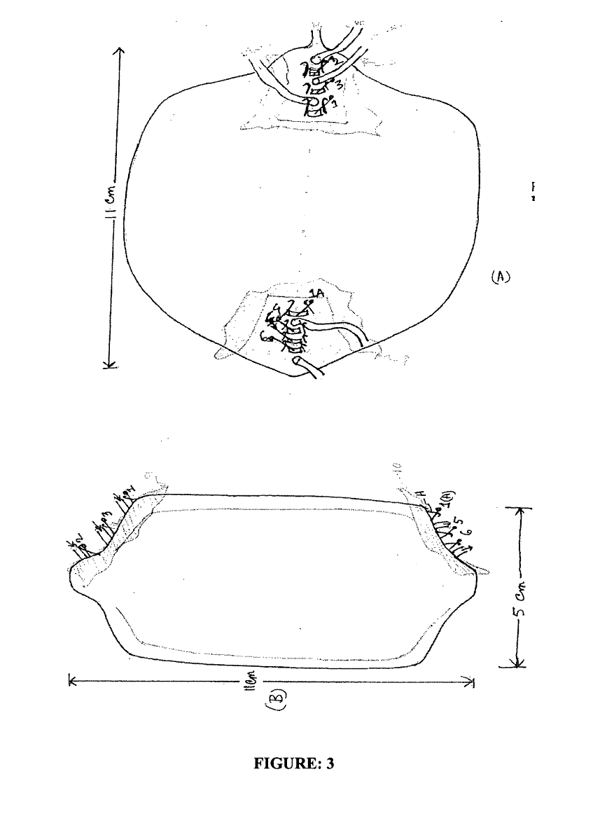

There is a chamber for storing L1 solution, in which L1 solution comes after passing through a bubble trapper valve or symmetrical system which never permits any air bubble to enter in the chamber 26. Here we should remember that the volume of the stored L1 solution should be at least two to four times more of the inner volume (capacity) of the membrane. The L1 enters the path 11A & B respectively, which is controlled by knob 1. Which allows at a time L1 can either enter 11A or 11B (but never both). In some cases 7, 8, 9, 10 and valve 6 can also be removed, where the subject cant have the controlled ultra-filtration facility. In membrane we can observe drainage passage, marked as D1, D2 respectively. The membrane may be single or plural accordingly. The shape of the system as shown in the FIG. 3 (but it may be of any other shape). In FIG. 3 an extra texture is observed, which is marked as 7, 8 (in top view) and 9, 10 (side view). These made of bio-compatible polymers or any other tissue cultured/artificially developed cells. These resist maximum infection, bleeding because of external connection. These extra texture can be added (by plastic surgery) easily with skin. According to the present model, the L1, L2 solutions are required to supply externally (in case of implantable system, again in some cases the waste materials can be sent out directly through 19 [according FIG. 1]. The shape, size of the cover (14), chamber (15, 18) and all channels (11, 11A, 11B, 13, 19A, 19B, 20, 20A, 20B) can be made of any material which is safe to human body and can be altered in shape as per the requirement.

### Example: 7

A circulating programmable and adjustable (battery operated) small pump (programmed variably with respect to time using any microprocessor/any other circuitry) can also be fixed with the 20 or respectively with 20A & 20B; and also with 19. This will help for quick clearance of the wastes and also high security for both the system & subject with high performance. (“S” type blood circulator is already available in market).

We can also eliminate the other valves (except 27, 28) with unipolar/bipolar (example: “V” shaped) circulator. In case of unipolar or “V” shaped circulator, if the contacting rod/shaft is touched (and stopped by controlling them) with the corresponding channels, the circulation is blocked through that channel, on other hand when it rotates, the circulation and also the increased clearance rate might be achieved (without of any manual controlling).

In case of the battery operated system (circulatory based) the extra texture or polymeric mass of the valves (except 27, 28) inside the channel might be omitted but care should be taken that the circulator is providing the desired functions. Knobs or handles are also not required to control manually or may be kept optionally in this case.

In case of external use of the system, only two or three (preferably 2) outlets are required to attach the system with human subject. Here, one catheter is required to make the outlet of unpurified blood; another one catheter is for sending purified blood in the subject. Metabolic wastes are recommended to send out directly from artificial kidney system.

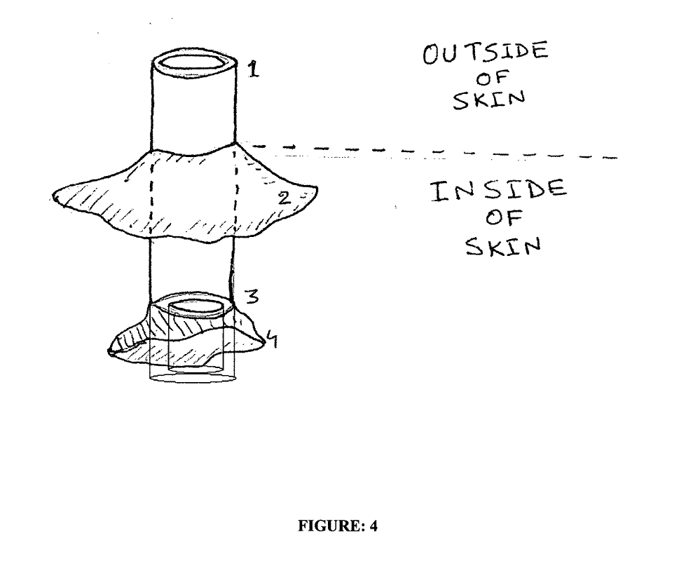

The catheter (both for implantable usage/external usage) can be observed in FIG. 4. Here, a pipeline/catheter is made up of bio-compatible polymers and can be permanently fixed with renal artery/any other arteries according the recommendation of the doctors (like heart bypass surgery, etc.). A flexible polymeric extra texture is observed, marked as 2 & 4; which can be directly stitched (surgery) with the natural blood artery/vein accordingly. This special type of permanent implantation of catheter provides a long life time of the blood connection paths, removes the threatening of infections and other hazards. Again, the extra polymeric flexible structure 2 according FIG. 4, provides a wide range of security because of commonly used catheters. This part 2 is to be attached with skin by plastic surgery and proper dressings.

FIG. 2 show several membranes. We use preferably “V” shaped paper type membrane. The shape, size, number of the membrane can be varied and not limited accordingly of the diagram.

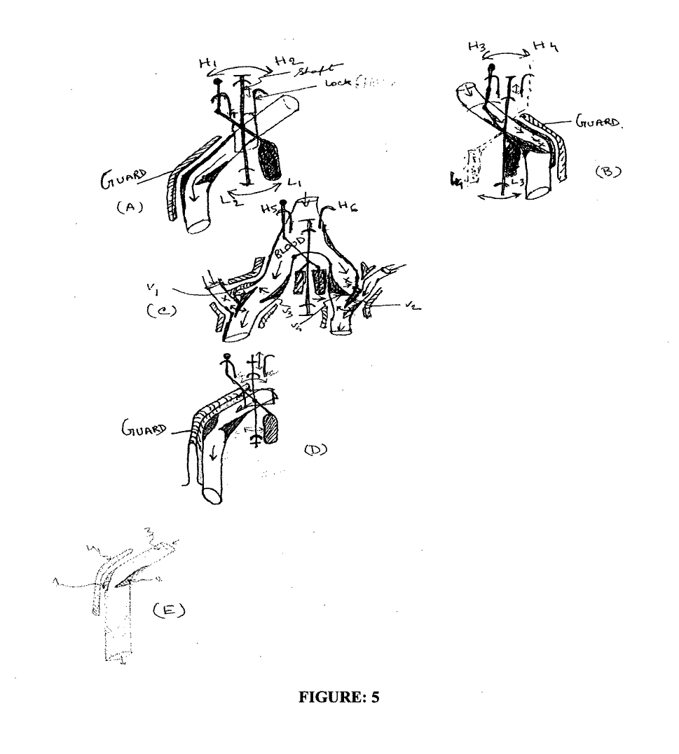

FIG. 5 disclose the control systems for the different valves. A skeleton of nano-carbon/metallic net/stain of different valves might be observed in the FIG. 5E. To prepare the desired valves/vessels we can put extra mesh on the portion 1 & 2 of the skeleton according the FIG. 5E. The rest procedure is simple and similar of preparing existing blood vessels (by dipping the skeleton in the liquid polymer & then doping/embedding them with therapeutic drugs to obtain desired specification and bio-compatibility). Even the normal existing blood vessels can be used, but the place of the wall should be much thick so that it should not damaged by continuous knob operation. All valves, apart from 1, 8, and 25 are of this type.

In FIG. 5A it can be observed that the handle/knob H have 2 positions (which is obtained by changing the position of H1-H2) and can be locked using a hook or any other locking system to get desired function. In this locking system, the movable shaft (FIG. 5A) is attached to the inner wall of the cover (14, in according FIG. 1). Altering the position of H1 and H2, the position of L1 and L2 also changes, which leads the opening and closing of the path as desired. Example: in FIG. 5A, the handle position H1 allows blood to flow through the vessel, and H2 closes the vessel. The fixed un-movable guard as shown in fig must be of any hard material and should be fixed in the system. Again FIG. 5C discloses another kind of multiple valve operation used for controlling L1 and Unpurified blood through membrane. It can be observed that, because of position H5 of this valve, the valve V3 is in relaxed condition and hence allowing the blood to flow through this path, and hence the valve V1, which controls the flow of L1 solution, is blocked. Again, the same position of the knob H5 presses the valve V4, which blocks the blood flow through the vessel. Locked position of V4 touches the bottom part of the mounted valve V2, which indeed open the path of L1 solution. Hence in this position, through this vessel only L1 solution can enter in the membrane. Any other kind of valve(s)/controlling systems facilitating similar operation or function can also be used in place of the present valves. Like, Variable Regulator knob type valves & knobs may also be used in it.

### Example: 8

Functions and Working of the Blood Purification Apparatus or Artificial Kidney

The total system (ARTIFICIAL KIDNEY) consists of 2 identical chambers made of pure specially treated (medicated) stainless steel/any other bio-compatible materials, which neither react with blood nor harm our body along with a membrane placed in each chamber and the same is shown in FIG. 1. A point to be noted is that at a time each valve is able to lock/un-lock only one side of the channel, in which it is connected. The working of the total system is divided in two parts:
i) Filtration cycle or dialysis
ii) Refreshment cycle

FIG. 1, the ultra purified blood is coming through channel 11, which is again divided in two parts (11A & 11B). Now as described earlier, suppose the unpurified blood is entering channel 11A (by controlling the knob 1 and placing it to H1 position). So, through 11A only unpurified blood is entering the membrane (17), where as because of this position of valve 1, only L1 solution is entering the channel 11B, and membrane 16. Hence its clear that blood is flowing through the membrane 17 and L1 solution is in the membrane 16. So, the chamber A is in dialysis cycle along with its membrane; where as the chamber B is in refreshment cycle with its corresponding membrane.

In refreshment cycle, L2 (dialysate) solution is entering to the chamber B by opening controlling knob 3. At the same time we keep the knob 5 in open position. Now the chamber B is filled with the L2 solution. The membrane (16 and 17) placed in this chamber (15, 18) contains L1 solution, which has very lower density than L2. Hence a backpressure is created on the membrane (16 and 17), which clears the blocked pores of membrane (which are blocked/obstructed because of forward pressure in dialysis cycle), and regain the efficiency/porosity of the membrane, at the same time it does not allow the membrane (16 and 17) to reach its saturation level. And hence the membrane can be reused many times. To attain this cycle, the knobs (1A and 5) should kept in the lock position, which does not permit L1 or L2 solution to come out from the system.

After finishing this cycle, the knob (1) is changed its position and allows unpurified blood to flow through the system, and starts dialysis cycle for this chamber (15, 18) and corresponding membranes (16, 17) placed in it. At the same time knob (5) is also required to alter its position to let the blood sent in the circulation system. This allows the L1 solution to go to the blood circulation system of the body of the subject. The unpurified blood has very much higher density than the moderated L2 solution (moderated L2 solution=after finishing the Refreshment cycle the density of L2 is decreased). Here in dialysis cycle, the blood has high pressure too. Which increase the clearance efficiency of the system to clear metabolic wastes from unpurified blood, and hence purifies the blood. After finishing this cycle again we need to alter the positions of the knobs & valves to restart dialysis cycle of that chamber/membrane.

When one membrane (16, 17) /chamber (15, 18) is in dialysis cycle, the other one is in refreshment cycle. This process allows the blood purification process throughout 24 hours (12 hr+12 hr). One attempt was made, by connecting the channel (10) directly in place of inlet L1 solution through channel (13) (eliminating, 25, 26, 13, 10 and valve 6) which was able to provide a high reliability as no provision of air bubble to enter the blood circulation system. But that was having several back logs, as no ultra filtration can occur there; again there was no provision to inject therapeutic drugs or any other. required medicine to enrich its property and to balance body chemistry.

For its external use, we can also use single membrane (16, 17) /chamber (15, 18). In that case, there is no provision of refreshing cycle. Such system can be applied to the patients with very lower requirement of dialysis process (like initial stage of renal failure). And after use of the membrane for some fixed time period/some cycles, the membrane (16, 17) is needed to be changed. Such system has low life time and decreased efficiency if not used properly.

A cover of stainless steel/any other metals/materials or alloy can be used here, which is doped in and coated with therapeutic drugs as discussed in U.S. Pat. No. 5,749,880. Such approach eliminates the hazards of using the metallic materials in subjects.

### Example: 9

Procedure to Send the Urine to the Bladder:

Here we prefer to have an automatic battery operated timer based control system which can be fixed outside of the body; this will increase the efficiency of the whole system. The handles, channels (entering the body) should be fixed in the body by medical operation/surgery such that it won't harm our body, means blood and other body elements should not come outside of the body, and also the patient should not feel pain when the handles will be altered. The chamber (26) can be placed outside of body one filled with L1 solution, and the other chamber with L2 solution which is not shown in the FIG. 1, both of these containers are connected to its respective channels (13, 12) respectively, through knobs (2, 3) properly. The chamber (26) containing the solution L1 and chamber for L2 solution not shown in FIG. 1 has to re-fill each day—regularly.

If one kidney is damaged then also we have to place the whole system inside the body, to keep the other kidney as it is, we have to connect the whole system with the artery of damaged kidney, and the purified blood will be sent to the vein of the same (damaged) kidney, and then we have to change the timings accordingly.

The mechanical system is controlled and should be fixed in the body by proper surgery, in such a way so that no blood/other body materials can come outside of body. If the control is manual process, the person should be careful about timing to alter the positions of handles (H1, H2).

If the control system is battery operated external mechanical system, care should be taken to check whether the controlling machine is working properly or not. Proper care should be taken about battery also. Being a complex system (artificial kidney, with L1 and L2), its heavier than our original kidney. Care should be taken to fill up the re-fill containers by L1 and L2 respectively.

The complete system is efficient to do the job of original kidney in our body, without causing any harm of our body; balancing properly the chemistry of blood and at the same time of our body, having the merits of it; if required we can use this artificial kidney instead going for general dialysis. If both the kidneys are damaged, then also the patient can survive by using this artificial kidney system. The shapes, size of the containers containing L1 and L2 solution and the timing to control the valve-channels can be varied uniformly as per the requirement of patient.

### Example: 10

Experimental Studies

Blood Test: Animal Trial:

TABLE 2
Blood test data in animals such as dog, goat and sheep

| | Dog | | Goat | | Sheep | |
|---|---|---|---|---|---|---|
| Final system.: (after modification) | B.A. | A.A. | B.A. | A.A. | B.A. | A.A. |
| Blood Glucose: (unit --mg/100 ml blood) | 78 | 82 | 48 | 53 | 52 | 54 |
| Serum Protein: (unit --mg/100 ml blood) | | | | | | |
| Alpha Globulin: | 1.21 | 1.217 | 0.43 | 0.434 | 1.12 | 1.114 |
| Beta Globulin: | 1.32 | 1.33 | 1.25 | 1.258 | 0.45 | 0.452 |
| Gamma Globulin: | 0.86 | 0.87 | 0.9 | 0.91 | 1.31 | 1.32 |
| Albumin | 3.3 | 3.32 | 3.89 | 3.92 | 3 | 3.03 |
| Serum Calcium: (unit --gm/100 ml blood) | 9.8 | 9.82 | 10.68 | 10.72 | 11.39 | 11.44 |
| Serum Inorganics Phosphorus (mg/100 ml blood)* | 4.2 | 3.1 | 5 | 3.8 | 6.8 | 5.21 |
| Serum Magnesium (mg/100 ml blood) | 2.1 | 2.12 | 3.28 | 3.26 | 2.51 | 2.512 |
| Serum Cholesterol (mg/100 ml blood) | 122 | 122 | 94 | 95 | 71 | 71 |
| Serum Creatinine (mg/100 ml blood) | 9.2 | 1.3 | 8.3 | 1.15 | 8.1 | 1.4 |
| Serum Urea (mg/100 ml blood) | 52 | 12 | 64 | 15.6 | 50 | 10.1 |
| Serum Chloride ((mEq./Lt)* | 108 | 109.1 | 116 | 118.3 | 101.2 | 99.8 |
| Serum Sodium (mEq./Lt)* | 145 | 148 | 148 | 149 | 149 | 153 |
| Serum Potassium (mEq./Lt)* | 4.1 | 4.3 | 3.6 | 3.65 | 4.9 | 5 |

Note:
Here, B.A = Before Application of Artificial Kidney system/before surgery; by inducing renal failure of the subject for system testing and analysis;
A.A = After application (8 hours) of Artificial kidney system/after surgery; in induced renal failure condition. Experimental uremia has been induced by uretral litigation or clipping of renal artery.

Blood Test: Human Trial:

TABLE 3
Blood test data in Humans

| | TIME OF APPLICATION | | | | | | | |
|---|---|---|---|---|---|---|---|---|
| | BEFORE APPLICATION | | | | AFTER APPLICATION | | | |
| | SEX AND AGE OF THE SUBJECT | | | | | | | |
| | MALE 52 | | FEMALE 58 | | MALE 52 | | FEMALE 58 | |
| Blood Urea Nitrogen (BUN)* | 39.6 | mmol/L | 48.8 | mmol/L | 2.5 | mmol/L | 2.4 | mmol/L |
| Serum Creatinine* | 344 | μmol/L | 432 | μmol/L | 52 | μmol/L | 58 | μmol/L |
| Serum Bilirubin | 0.4 | gm/dl | 0.3 | gm/dl | 0.4 | mg/dl | 0.3 | mg · dl |
| Serum Albumin | 3.8 | gm/dl | 3.8 | gm/dl | 3.8 | gm/dl | 3.8 | gm/dl |
| Serum Globulin | 2.8 | gm/dl | 2.7 | gm/dl | 2.8 | gm/dl | 2.7 | gm/dl |

Serum Cholesterol: (by WYBENGA et al. method)

| | | | | | | | | |
|---|---|---|---|---|---|---|---|---|
| High Density Lipoprotein | 0.73 | mmol/L | 0.99 | mmol/L | 0.73 | mmol/L | 0.79 | mmol/L |
| Cholesterol (HDL) | | | | | | | | |
| Low Density Lipoprotein | 2.34 | mmol/L | 2.31 | mmol/L | 2.34 | mmol/L | 2.34 | mmol/L |
| Cholesterol (LDL) | | | | | | | | |
| Triglycerides | 0.12 | mmol/L | 0.13 | mmol/L | 0.12 | mmol/L | 0.13 | mmol/L |
| Blood Glucose | 3.5 | mmol/L | 3.6 | mmol/L | 3.6 | mmol/L | 3.8 | mmol/L |
| Serum Calcium | 2.28 | mmol/L | 2.29 | mmol/L | 2.28 | mmol/L | 2.29 | mmol/L |
| Uric Acid* | 461 | μmol/L | 482 | μmol/L | 182 | μmol/L | 191 | μmol/L |
| Inorganic Phosphorus* | 2 | mmol/L | 2.2 | mmol/L | 0.90 | mmol/L | 0.91 | mmol/L |
| Chloride* | 98 | mmol/L | 97.9 | mmol/L | 101 | mmol/L | 101 | mmol/L |
| Serum Iron | 125 | μg/dl | 142 | μg/dl | 130 | μg/dl | 144 | μg/dl |
| Total Iron Binding | 285 | μg/dl | 301 | μg/dl | 288 | μg/dl | 303 | μg/dl |
| Capacity (TIBC) | | | | | | | | |
| Serum Magnesium | 2.066 | mg/dl | 2.236 | mg/dl | 2.28 | mg/dl | 2.40 | mg/dl |
| Serum Alkaline Phosphatase | 68 | U/L | 72 | U/L | 64.43 | U/L | 66.2 | U/L |
| Lipase Serum | 2.34 | μKat/L | 1.1 | μKat/L | 2.2 | μKat/L | 1.6 | μKat/L |
| Acid Phosphatase (PNPP | 7.2 | μKat/L | 6.2 | μKat/L | 7.4 | μKat/L | 5.9 | μKat/L |
| Method) | | | | | | | | |
| Serum Analine Aminotransferase | 34 | U/L | 28 | U/L | 31 | U/L | 27.6 | U/L |
| Asperate Aminotransferase | 0.29 | μKat/L | 0.18 | μKat/L | 0.27 | μKat/L | 0.19 | μKat/L |
| Lactic Dehydrogenase (LDH) | 72 | U/L | 76 | U/L | 71 | U/L | 76 | U/L |
| Potassium* | 3.2 | mmol/L | 3.4 | mmol/L | 3.6 | mmol/L | 3.7 | mmol/L |
| Sodium* | 137 | mmol/L | 138 | mmol/L | 138.2 | mmol/L | 138.5 | mmol/L |

*The test conducted continuously for more than six months with the result ±2-4% change of the above mentioned data. Both the subjects were under renal treatment because of their kidney failure. Here the system was attached to their arm (externally) so that if require, the system/subject can be managed/rectified properly at any instance of the treatment.

For biocompatibility testing, the product is tested in dog, goat and sheep. It is clearly observed that the subject urea, creatinine level etc were drastically decreased on the application of the system successfully. Here, after application indicates after application of the artificial kidney device, after 8 hrs.

The detailed report of altering parameters to evaluate the system efficiency is disclosed in the below Table: 4 and 5 for male and female subjects separately.

TABLE 4
Evaluation of system efficiency in male subjects

| | | TIME IN HOURS | | | | | |
|---|---|---|---|---|---|---|---|
| Male Subject: | Unit: | 0 | 2 | 4 | 6 | 8 | 12 |
| Blood Urea | mmol/L | 39.6 | 24.2 | 11.4 | 2.9 | 2.5 | 2.4 |
| Nitrogen | | | | | | | |
| (BUN)* | | | | | | | |
| Serum Creatinine* | μmol/L | 344 | 263 | 174 | 82 | 52 | 52 |
| Uric Acid* | μmol/L | 461 | 386 | 312 | 245 | 182 | 181 |
| Inorganic | mmol/L | 2 | 1.7 | 1.5 | 1.3 | 0.9 | 0.9 |
| Phosphorus* | | | | | | | |
| Sodium* | mmol/L | 137 | 137.5 | 137.7 | 137.9 | 138.1 | 137.6 |
| Potassium * | mmol/L | 3.2 | 3.3 | 3.4 | 3.6 | 3.6 | 3.4 |
| Chloride* | mmol/L | 98 | 98.5 | 98.9 | 100.2 | 99.9 | 99.9 |

TABLE 5
Evaluation of system efficiency in female subjects

| | | TIME IN HOURS | | | | | |
|---|---|---|---|---|---|---|---|
| Female subject: | Unit: | 0 | 2 | 4 | 6 | 8 | 12 |
| Blood Urea | mmol/L | 48.8 | 28.2 | 13.3 | 3.2 | 2.5 | 2.5 |
| Nitrogen (BUN)* | | | | | | | |
| Serum | μmol/L | 432 | 320 | 180 | 86 | 58 | 58 |
| Creatinine* | | | | | | | |
| Uric Acid* | μmol/L | 482 | 391 | 319 | 254 | 191 | 185 |
| Inorganic | mmol/L | 2.2 | 1.8 | 1.5 | 1.3 | 0.91 | 0.9 |
| Phosphorus* | | | | | | | |
| Sodium* | mmol/L | 138 | 137.8 | 137.9 | 138 | 138.2 | 138.3 |
| Potassium* | mmol/L | 3.4 | 3.5 | 3.6 | 3.6 | 3.7 | 3.7 |
| Chloride* | mmol/L | 97.9 | 98.3 | 98.9 | 100.1 | 98.8 | 99.8 |

From the detail report of altering parameters to evaluate system efficiency: Male/Female Subject] From table 4 & 5 it can be clearly observed, there is a massive change in case of BUN, Creatinine, Uric acid; whereas a little alteration in Chloride, Sodium, and Potassium. Here Inorganic phosphorus is also becoming normal after its 8 hours use. Here it should be noted the Urea, Uric acid, Creatinine are also becoming normal by that time. If the process is continued for some more hours, then also there is no problem of all these parameters in blood.

Separation of Dissolved Waste Materials From the Simulated Blood:

TABLE 6
Separation of waste materials from the simulated blood using the
apparatus of instant invention

| | CLEARANCE TIME IN HOURS | | | | |
|---|---|---|---|---|---|
| Name of the | 0 | 2 | 4 | 6 | 8 |
| Substance | (mg) | (mg) | (mg) | (mg) | (mg) |
| UREA: | | | | | |
| without absorbent | 830 | 1655 | 3300 | 4520 | 5100 |
| with absorbent | 885 | 1766 | 3520 | 4800 | 5800 |
| CREATININE: | | | | | |
| without absorbent | 105 | 195 | 410 | 578 | 720 |
| with absorbent | 138 | 266 | 540 | 786 | 790 |
| URIC ACID: | | | | | |
| without absorbent | 38 | 72 | 148 | 196 | 210 |
| with absorbent | 49 | 94 | 192 | 250 | 260 |
| PHOSPHORUS: | | | | | |
| without absorbent | 36 | 69 | 135 | 184 | 220 |
| with absorbent | 45 | 88 | 175 | 220 | 298 |
| VITAMIN B12: | | | | | |
| without absorbent | 3.9 | 7.6 | 15.3 | 18.6 | 24 |
| with absorbent | 5.1 | 9.8 | 19.8 | 22.5 | 29 |

The hours mentioned above in the clearance chart is indicating the time of alteration/continuity of the dialysis/refreshment cycle. If the continuation of dialysis cycle exceeds 8 hours, then the system efficiency is decreased drastically; in such case, the membrane can't be refreshed by normal temperature/pressure or normal recycling mechanisms and need steam, other refreshing chemicals & corresponding processes as seemed appropriate. The above data are with nano-carbon net polymeric membrane. In case of only polymeric membrane, the data were decreased by 20% than that of the above data.

The present system is simulated using the simulated blood compositions and the details of the same are provided in table: 7

TABLE 7
Details of simulated blood composition

| | Concentration |
|---|---|
| Simulated Blood: Composition ingredients | in mg/dl |
| Urea: | 180 |
| Creatinine: | 20 |
| Uric Acid: | 14 |
| Phosphorus: | 6 |
| (Mixture of Na.sub.2 HPO.sub.4 + | |
| NaH.sub.2 PO.sub.4) | |
| Vitamin B12: | 2.5 |

The waste solution coming out of the system after dialysis is nothing but urine which is considered for analysis to estimate the elimination potential of the apparatus. The urine obtained is tested for various waste materials using conventional methodologies and the results are tabulated in the below table: 8.

TABLE 8
Analytical data of the waste material obtained
using the apparatus of the instant invention

| Name of the substance | Unit: | 1st Day | 2nd Day | 3rd Day |
|---|---|---|---|---|
| Chlorides:* | gm/dl | 0.7 | 0.68 | 0.69 |
| Sodium:* | gm/dl | 0.6 | 0.57 | 0.53 |
| Calcium:* | gm/dl | 9 | 9.5 | 10 |
| Phosphate | gm/dl | 0.125 | 0.128 | 0.129 |
| Urea | gm/dl | 1.9 | 1.8 | 1.86 |
| Creatinine | gm/dl | 0.13 | 0.128 | 0.13 |
| Uric Acid | gm/dl | 0.04 | 0.042 | 0.042 |
| Glucose* | gm/dl | 10 | 8.8 | 11 |
| Potassium* | mmol/L | 0.9 | 0.8 | 0.8 |

It can be clearly observed that some parts of sodium, calcium, potassium and glucose are coming out with the solution after dialysis, but from the blood test it can be seen that there is no alteration in the property/characteristics of blood because of these. Furthermore, the presence of these particles (* marked in above table:8) in the waste solution is due to their usage in L2 solution.

### Example: 11

Required/Recommended Blood Pressure for the Effective Working of the Present Invention:

The required blood pressure of the subject is preferably 40-200 mm Hg. whereas it is also observed that the system can work (less effectively) in lower blood pressure. Higher blood pressure indicates hazards to subject of coronary thrombosis & others. Though, the present invention can be effectively operated in higher blood pressure. For effective & super performance of the present invention it is advised to maintain a balance blood pressure within normal range. Balanced blood pressure also maintains all other physiological body chemistry for a healthy-balanced life.

Lifetime of the Present Invention on Its Effective Application:

The quality of the urine and the blood purification quality proved the efficiency of the present invention around 95% compared to original ideal natural kidney. Average case was considered to study the longevity of present invention. We made blood flow through the system around 0.5 liters/min and the blood pressure for the same we kept around 80/120 mm pressure of Hg. Even in the case of high blood pressure around 120/200 mm pressure of Hg (which is one of the most important reasons leading to kidney failure). Even the present system was tested in low blood pressure in 30 to 180 mm pressure of Hg to study low pressure effect on the present invention. The valves were controlled (altered) in a very fast mode 22 times/minute. Here we got the actual and encouraging reports for 1182800 liters of blood purified by the kit. It is a very common clinical data that our kidneys purify around 180 liters/24 hours. Hence from the obtained result for the accurate case we obtained the longevity of Present invention is around 18 years (approximately in average). The same tests were conducted on 5 artificial systems with symmetrical setup.

After 18 years, the present invention can be replaced with another symmetrical system and the subjects are hereby recommended to undergo regular pathological checkup (at least urine and blood to measure the effectiveness of the present invention). If accidentally any disorder found in the reports, doctors can replace the present/malfunctioning system with another one.

### Advantages of the Present Invention

1. It is efficient and sufficient to do all the job of original-natural kidneys (except ERYTHROPOIETIN secretion) for 24 hours, all the days, throughout the years (approximately 18 years in average in average case).

2. It is irrespective and independent of blood group and all other corresponding parameter leading to renal/kidney transplantation of the subject (like tissue matching).

3. There won't be any problem if both the original natural kidneys of the patient are not working properly or damaged, we just need to replace/transplant (without replacing natural mal functioning kidneys) the original natural kidneys with the ARTIFICIAL KIDNEY system. Else for single kidney failure we can do the transplantation of the ARTIFICIAL KIDNEY for the single malfunctioning kidney.

4. The system can be placed inside of our body (might be by pocket surgery, or as desired by subject and doctors). It can also be attached to our body externally (portable) and blood connections needed to make properly in such case providing all sorts of security & risks measures.

5. The system is less hazardous as no air bubble can enter in the blood circulation system (which is a risk for general normal dialysis both the haemodialysis and peritoneal dialysis). The ARTIFICIAL KIDNEY is effective enough to meet all the requirements of normal haemodialysis and peritoneal dialysis.

6. The chemicals used in the system are sufficient to balance the properties of blood and at the same time the whole body chemistry.

7. It is not painful for the patient like general dialysis system, further more actually there is no permanent cure for general dialysis and subject (patient) need to go through the same process after some time span and need to repeat the same procedure, and if both the original kidneys fail then these general dialysis cant be execute at all, which have been overcome by the present invention: ARTIFICIAL KIDNEY. The artificial kidney having a higher life span (as the complete system is divided in two parts: DIALYSIS CYCLE and REFRESHMENT CYCLE, having just opposite direction with respect to other cycle).

8. Less dependability to medical practitioners, hospitals.

9. There is no need of any expert person for its regular use. The elements used in the system are not harmful at all for our body.

10. As the purification process of the blood happens here in continuous basis, the error elements (metabolic=urea, uric acid, creatinine, Phosphate, etc.) will let out from the body of subject (patient) in the form of urine. And hence all the living cells of our body get refreshed by the circulation of fresh blood all the time in our body. As a result the life spans of the cells in our body also increased to an ultimate extend. The system at the same time also provides the guarantee that not a single blood cell (i.e. RBC,WBC,etc.) will come out with urine, etc.

11. We can directly put some venous drugs directly through L1 solution, or some drugs with L2 solution which is permeable through the membrane (bio-compatible & non reactive to the membrane) as per the requirement of the subject.

12. No need of a donor's kidney.

### Reference Books: Non-Patent Literature

1. Arthur C. Guyton, John E. Hall; “Text Book of Medical Physiology” Eleventh Edition, Saunders, 2006, pp. 307-314, 419-467.
2. N. V. Bhagavan, “Medical Biochemistry” Fourth Edition, Academic Press, 2002 pp. 1-16, 840-934.
3. Editor: T. J. Greenwalt, “Methods in Hematology BLOOD TRANSFUSION”, Volume 17, Churchill Livingstone, 1988, pp. 1-305.
4. Jeremy Levy, Julie Morgan, Edwina Brown; “Oxford Handbook of Dialysis”; Oxford University Press, 2005, Indian Edition, pp. 4-878.
5. John G. Webster, “Medical Instrumentation Application and Design”, John Wiley & Sons, 2004, Wiley Student Edition, pp. 600-603.
6. Katona, P. G., “Drug Infusion System”; in J. G. Webster edition, “Encyclopedia of Medical Devices and Instrumentation”, New York, Wiley, 1988, pp. 971-980.
7. Langer, R., “1994 Whitaker Lecture. Polymers for drug delivery and tissue engineering.” Ann. Biomed. Eng., 1995, 23, 101-111.
8. D. Jennings, A Flint, B C H Turton, L D M Nokes, “Introduction to Medical Electronics Application”, Edward Arnold, 1995, pp. 1-95, 204-213.
9. Editors: P. G. Urben, Azko-Nobel, Compiler: M. J. Pitt, “Bretherick's Handbook of Reactive Chemistry”, SIXTH EDITION, Volume 1, Butterworth-Heinemann, 1999, pp. 1539, 781, 828, 833, 764, 1407, 831, 6291, 417, 1919, 2669, 3262, 782, 1108, 4831, 1221, 1728, 1101, 1703.
10. Editors: K. Tanaka, T. Yamabe, K. Fukui †, “The Science and Technology of Carbon Nanotubes”, Elsevier, 1999, pp. 2-86, 128-143.
11. Editors: Buddy D. Ratner, Allan S. Hoffman, Frederick J. Schoen, Jack E. Lemons, “Biomaterial Science”, Academic Press, 1996, pp. 1-470.
12. Editors: D. A. Dillard, A. V. Pocius, “Adhesion Science and Engineering-1”, Elsevier B V, 2002, pp. 1-442.
13. Editors: Simon Judd, Bruce Jefferson, “Membranes for Industrial Wastewater Recovery and Re-use”, Elsevier, 2003, pp. 2-75, 172-253
14. Milton Ohring, “The Materials Science of Thin Films”, Academic Press, 1992, pp. 1-589.
15. L. B. Freund, S. Suresh, “Thin Film Materials, Stress, Defect Formation and Surface Evolution”; pp. 56-339, 506-693.
16. Tiyong Tong, Adrian P. Mouritz, Michael K. Bennister, “3 D Fiber Reinforced Polymer Composites”, Elsevier, 2003, pp. 1-46.
17. Ruth Freitag, “Synthetic Polymers for Biotechnology and Medicine”, EUREKA.COM, 2003, pp. 1-157.
18. Wilfred L. F. Armarago, Christina Li Lin Chai, “Purification of Laboratory Chemicals”, FIFTH EDITION, Butterworth-Heinemann, 2003, 4-16, 56-388.
19. R. P. King, “Introduction To Practical Fluid Flow”, Butterworth-Heinemann, 2002, pp. 1-194.
20. A. S. Grandison and M. J. Lewis, “Separation Process In The Food And Biotechnology Industries; Principles and Applications”, Woodhead Publishing Limited, 1996, pp. 17-203.
21. Sami Matar, “Chemistry of Petrochemical Process”, 2nd Edition, Gulf Publishing Company, 2000, pp. 11-370.
22. Peter Smith, “Piping Materials Selection and Applications”, Elsevier, 2005, pp. 1-242.

23. Vitaly J. Klenin, “Thermodynamics of Systems Containing Flexible Chain Polymers”, Elsevier, 1999, pp. 1-99, 153-189, 385-427, 645-773.
24. Burton, C. V., “Pain suppression through peripheral nerve stimulation” in W. S. Fields and L. A. Leavitt (eds.). Nural Organization and Its Relevance to Prosthetics. New York Intercontinental, 1973, pp. 241-250.
25. “Handbook of Dialysis”, Fourth Edition, Editors: John T. Daugirdas, Peter G. Blake, Todd S. Ing; Lippincott Williams & Wilkins; a Walter Kluwer Business, ISBN: 81-89836-87-0.

### Patent Literature

### US Patents:

U.S. Pat. Nos. 5,855,601, 377,173, 3,939,069, 5,484,397, 5,336,165, 236,694, U.S. Pat. No. 6,361,562 B1, 5,783,124, 5,624,561, 5,580,568, 5,667,767, U.S. Pat. Nos. 7,138,106 B2, 6,451,048, 5,749,880, 5,855,600, 4,354,933, 5,957,508, 4,941,870, 5,483,172, 4,997,443, etc.

I claim:

1) An apparatus for purification of blood, wherein said apparatus comprising
a) membranes (16, 17) placed inside the chambers (15, 18) respectively, wherein chambers (15, 18) are located adjacently;
b) an impermeable jacket (9) having porous permeable membrane (9A) is connected to membranes (16, 17) through channel (11);
c) unidirectional valve (8) is connected to the porous permeable membrane (9A) to allow unpurified blood into the membranes (16, 17) using the channel (11);
d) a chamber (26) is connected to membranes (16, 17) through channel (13) to store L1 solution provided with bubble trapper valve (25) to prevent entry of air bubbles into the chamber (26);
e) outlets of chambers (18, 15) are connected to waste outlet (19) through channel (19A, 19B) respectively to carry impure L2 solution; and
f) outlets of membranes (17,16) are connected to purified blood outlet (20) through channels (20A, 20B) respectively to circulate purified blood to veins.

2) The apparatus as claimed in claim 1, wherein the flow of L2 solution into the chambers (15, 18) is controlled using knobs (2, 3).

3) The apparatus as claimed in claim 1, wherein knob (1) is used to control the flow of impure blood and L1 solution into the membranes (17, 16), and allows either the impure blood or the L1 solution into a membrane (17, 16) at any given time and maintains impure blood in any one of the membranes (17, 16) and L1 solution in the other membrane (17, 16).

4) The apparatus as claimed in claim 1, wherein knob (1A) is used to control the flow of pure blood from the membranes (17, 16) through the channels (20A, 20B) to the outlet (20) and opens one of the channels (20A, 20B) at any given time.

5) The apparatus as claimed in claim 1, wherein valves (4, 5) are used to control the flow of L2 solution from the chambers (15, 18) to the outlet (19) through channels (19A,19B).

6) The apparatus as claimed in claim 1, wherein valve (6) controls the removal of waste collected in impermeable jacket (9) through the outlet (19).

7) The apparatus as claimed in claim 1, wherein said membranes (16, 17) are biocompatible and are made up of polymers selected from a group comprising polyvinyl halides, polyurethanes, polystyrene derivatives, polyolefins, polyester series condensates, cellulose series high polymers and combinations thereof and said polyurethanes selected from a group comprising segmential polyurethanes and polyurethane urea.

8) The apparatus as claimed in claim 1, wherein said polymer membranes (16, 17) are sandwiched between single/multi layered nano-carbon/bio-compatible medicated metallic or non metallic (nylon or any other polymeric or other materials) nets and said are placed in “V” shape.

9) The apparatus as claimed in claim 1, wherein said channel (11) is divided into two sub channels (11A, 11B) to connect membranes (17, 16) respectively.

10) A method of assembling an apparatus for purification of blood, wherein said method comprising steps of:
a) placing membranes (16, 17) in chambers (15, 18) respectively;
b) connecting impermeable jacket (9) having porous permeable membrane (9A) to the membranes (16, 17) through channel (11);
c) connecting chamber (26) to the membranes (16, 17) through channel (13);
d) connecting outlets of the chambers (18, 15) to waste outlet (19) through channel (19A, 19B) respectively and connecting outlets of the membranes (17,16) to purified blood outlet (20) through channels (20A, 20B) respectively; and
e) mounting valves (4, 5) on to said channel (19A, 19B) respectively to control flow of waste and fixing knobs (1, 1A) to control flow of unpurified blood through channel (11) and purified blood through channels (20A, 20B).

11) The method as claimed in claim 10, wherein knob (2, 3) are placed to control flow of L2 solution through channel (12) which is connected to chambers (15, 18).

12) The method as claimed in claim 10, wherein valves (27, 28) are placed to control flow of L1 solution into membranes (17,16) through sub channels (11A, 11B).

13) The method as claimed in claim 10, wherein said polymer membranes (16, 17) are placed in “V” shape.

14) The method as claimed in claim 10, wherein said polymer membranes (16, 17) are sandwiched between single layered nano-carbon nets.

15) A method for purification of blood, wherein said method comprising steps of:
a) allowing impure blood to undergo coarse filtration through porous permeable membrane (9A) to remove the waste through outlet (19);
b) directing coarse filtered impure blood using knob (1) into one of the membranes (16 or 17) to undergo filtration while refreshing the other membrane (16, or 17) using L1 and L2 solution at a given time; and
c) collecting waste material into L2 solution after filtration and thereby removing the waste through the outlet (19) to obtain purified blood in outlet (20).

16) The method as claimed in claim 15, wherein the filtration of blood involves removal of waste such as urea, uric acid, creatinine and other metabolites.

17) The method as claimed in claim 15, wherein the refreshment of membranes (16 and 17) involves removal of impurities which are blocked inside the membranes (16 or 17) and maintaining L1 solution inside the membrane (16 and 17) and L2 solution outside the membrane (16 and 17) but inside the chamber (15, 18).

18) The method as claimed in claim 15, wherein said L1 solution is purified water with glucose and L2 solution is a mixture of sodium, potassium, chloride, calcium, magnesium, acetate/citrate, bicarbonate, glucose along with drugs belonging to the class of antiplatelets, anticoagulants, antifibrins, antithrombins, antiproliferatives, antiplatelets, anticouagulants, antifibrins, antithrombins and combinations thereof.

19) The method as claimed in claim 15, wherein said L2 solution has density always maintained higher than that of L1 solution to create back pressure on the membrane (16 or 17) so as to remove the blocked impurities.

20) The method as claimed in claim 15, wherein the membrane (16, 17) are sandwiched between single layered nano-carbon nets and are placed in “V” shape to create blood pressure and to achieve rapid filtration of blood.
